(19) United States
(12) Patent Application Publication
(10) Pub. No.: US 2011/0281893 A1
(43) Pub. Date: Nov. 17, 2011

(54) COMPOUND, SYNTHESIS, COMPOSITION AND USES THEREOF

(75) Inventor: Mahesh Kandula, Medapadu (IN)

(73) Assignee: KRISANI BIOSCIENCES (P) LTD, HYDERABAD (IN)

(21) Appl. No.: 13/103,174

(22) Filed: May 9, 2011

Related U.S. Application Data
(60) Provisional application No. 61/333,300, filed on May 11, 2010.

(51) Int. Cl. A61K 31/505; A61P 35/00; C07D 239/48

(52) U.S. Cl. 514/256; 544/329

(57) ABSTRACT

The instant application provides the disclosure for a compound of formula I, synthesis of compound of formula I and its pharmaceutical acceptable salts, as well as its polymorphs, solvates, and hydrates thereof. The pharmaceutically acceptable salt may be formulated for oral administration, systemic administration, transdermal administration and injection. The compound of formula I and its pharmaceutical compositions may be used to treat cancer in mammal.

### CROSS REFERENCE TO RELATED DISCLOSURE

This disclosure claims priority to pending U.S. Provisional disclosure 61/333,300 filed on 11 May 2010. This disclosure is hereby incorporated by this reference in their entireties for all of its teachings.

### FIELD OF DISCLOSURE

The instant disclosure generally relates to compound, synthesis of the compound and its pharmaceutical composition for the treatment of cancer. More particularly, this disclosure relates to treating mammals suffering from cancer with pharmaceutically acceptable dose of compounds.

### BACKGROUND

At present, cancer remains a significant health problem worldwide. According to International Agency for Research on Cancer-World Health Organization (IARC-WHO) estimates, cancer rates are set to increase at an alarming rate, from 10 million new cases globally in 2000 to 15 million in 2020.

The most common type of pharmacological anticancer treatment has been, for decades, conventional chemotherapy. These conventional treatments do not discriminate between rapidly dividing normal cells and tumor cells, thus leading to severe systemic side effects, while attempting to reduce the tumor mass. However, a significant unmet medical need still persists for the comprehensive treatment of cancer. There is currently a need in the art for new compositions for treatment of cancer.

### SUMMARY OF DISCLOSURE

The instant disclosure provides several embodiments for a compound, synthesis, composition comprising of formula I (Formula I) and/or pharmaceutical acceptable salts thereof. The disclosure also provides one or more compounds of formula I or intermediates thereof and one or more of the pharmaceutically acceptable carriers, vehicles or diluents as composition. These compositions (pharmaceutical or therapeutically effective) may be used in the treatment of cancer.

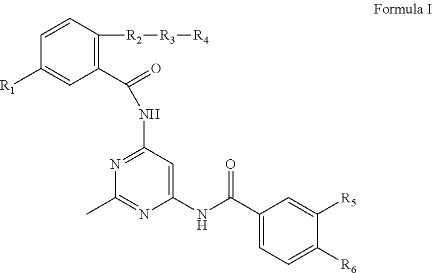

In certain embodiments, the present disclosure relates to the compound and composition of formula I, or pharmaceutically acceptable salts thereof,

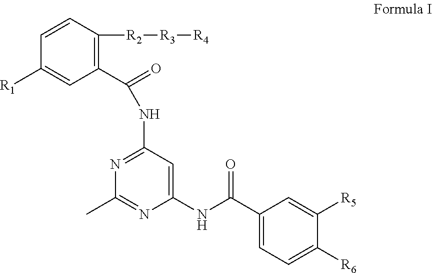

Wherein, R¹, R², R³, R⁴, R⁵, R⁶ each independently represents at least one of hydrogen, methyl, amine, cyclohexyl methyl ether, butoxy, propoxy, halogen (Chlorine or Flourine), thiol, alkyl, alkyl thiol, acetyl thiol, disulfide, acyl, acylalkyl, alkenyl, alkylthioalkyl, alkynyl, alkoxyaryl, alkoxyalkyl, aryl, aralkyl, aryloxyalkyl, arylthioalkyl, cycloalkyl, ether, ester, heteroaryl, heterocyclyl, lower alkyl, sulfone, sulfoxide, hydroxyalkyl, CF₃, CH₂Cl,

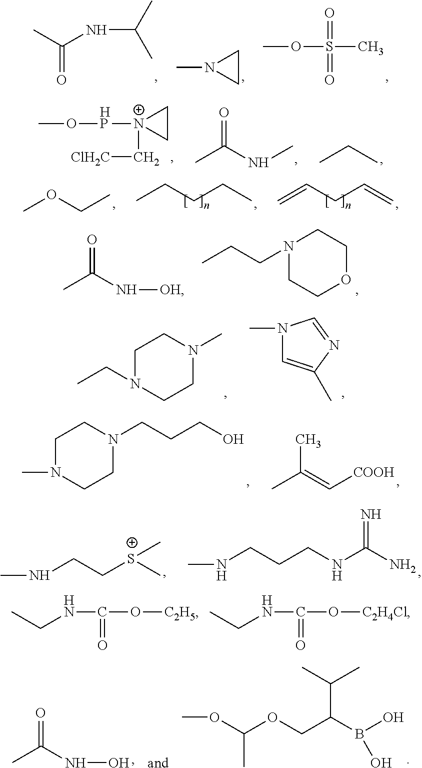

In certain embodiments, the present disclosure relates to the compounds and compositions of formula I or pharmaceutically acceptable salts thereof,

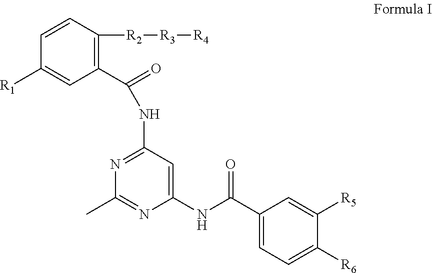

Wherein, R¹ independently represents at least one of

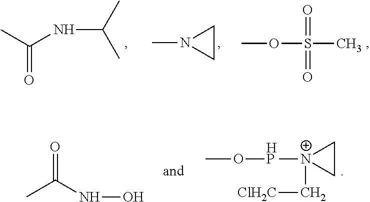

R² independently represents at least one of hydrogen, carboxyl, amine, —NH—CO—NH—, —NH—CO—CH₂—NH—,

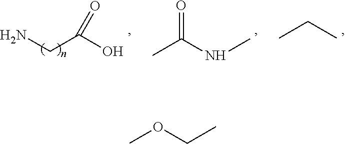

and an analog of any of the foregoing;
where, n represents an integer from 0 to 10
R³ independently represents at least one of hydrogen, methyl, ethyl, butyl, amine, —NH—CO—, acrolein,

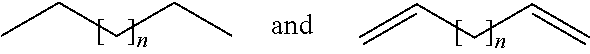

R⁴ independently represents at least one of

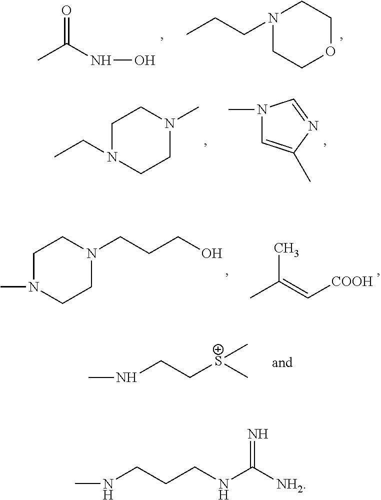

R⁵ independently represents at least one of CF₃ and (CH)ₙCl, where n is an integer ranges from 0 to 11
R⁶ independently represents at least one of

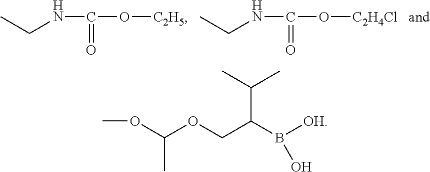

One embodiment of compound of formula I:

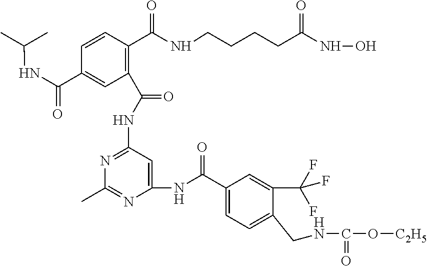

In one embodiment, synthesis of the compound of formula I is as shown in FIGS. 1, 2 and 3.

In one embodiment a kit comprising any one of the pharmaceutical compositions is disclosed herein. The kit may comprise of instructions for use in the treatment of cancer or related complications. The use of novel molecular targeted therapies is useful for both patients and clinicians. The treatment inhibits specific molecules that have a role in tumor growth or progression, and that are frequently altered in tumors but not in normal cells; thus, being more specific toward tumor cells, they are accompanied by reduced systemic toxicity. Instant compound and pharmaceutically effective dose may perform these functions.

In one embodiment, a pharmaceutical composition comprises a pharmaceutically acceptable carrier and any one of the compositions herein. In some aspects, the pharmaceutical composition is formulated and administered for at least one of for systemic administration, oral administration, sustained release, parenteral administration, injection, subdermal administration and transdermal administration.

The compositions described herein have several uses. The present disclosure provides, for example, methods of treating a patient suffering from cancer or malignant neoplasm which include Prostate cancer, Lung cancer, Colon cancer, Breast cancer, Brain cancer, Cervical cancer, Hodgkin's lymphoma, Kidney cancer, Leukemia, Liver cancer, Non-Hodgkin's lymphoma, Ovarian cancer, Skin cancer, Testicular cancer, Thyroid cancer or Uterine cancer.

### BRIEF DESCRIPTION OF FIGURES

Example embodiments are illustrated by way of example and not limitation in the figures of the accompanying drawings, in which like references indicate similar elements and in which:

FIG. 1A shows initial steps of synthesis of compound of formula 1.

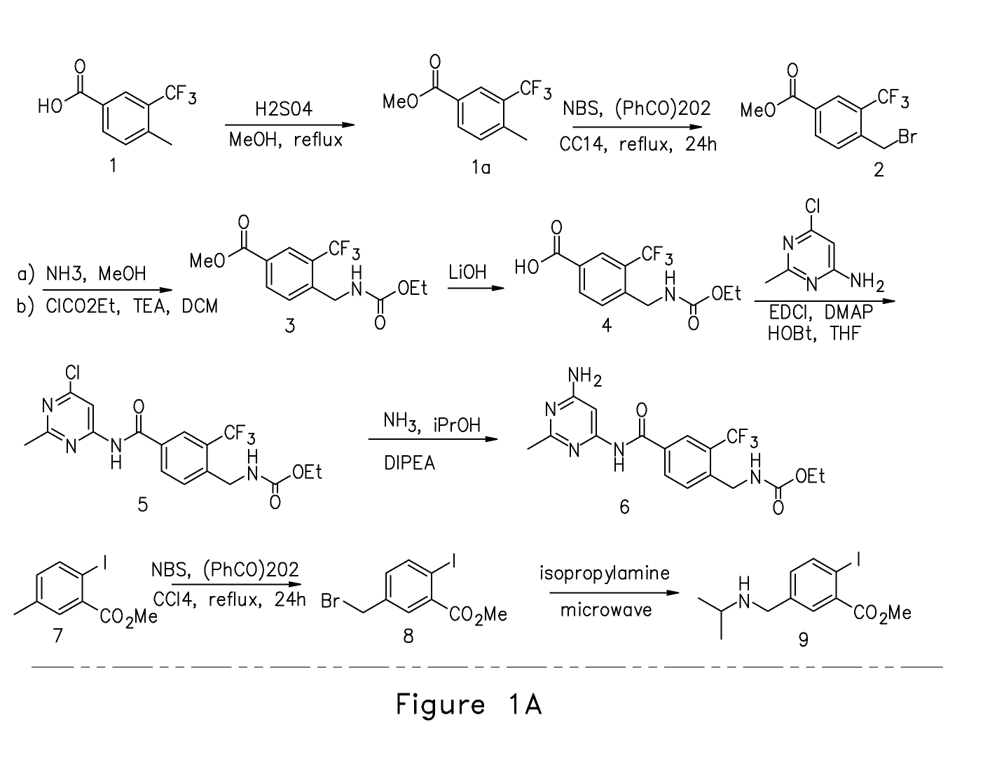

FIG. 1B shows continuation of steps from FIG. 1A for synthesizing compound of formula 1.

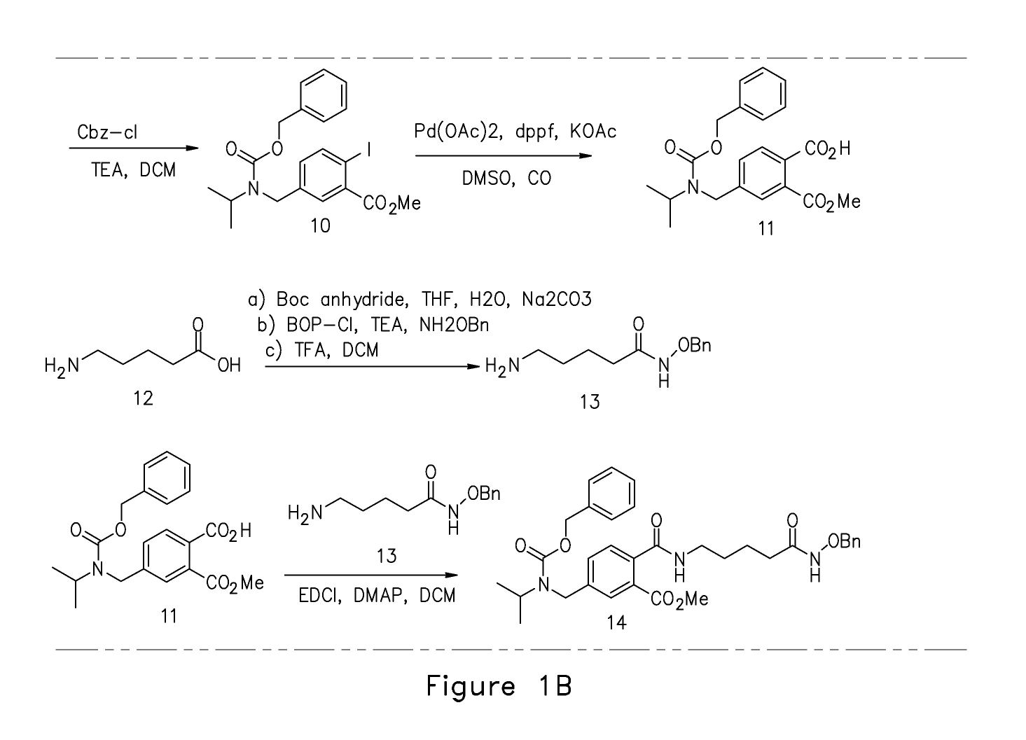

FIG. 1C shows continuation of steps from FIG. 1B for synthesizing compound of formula 1.

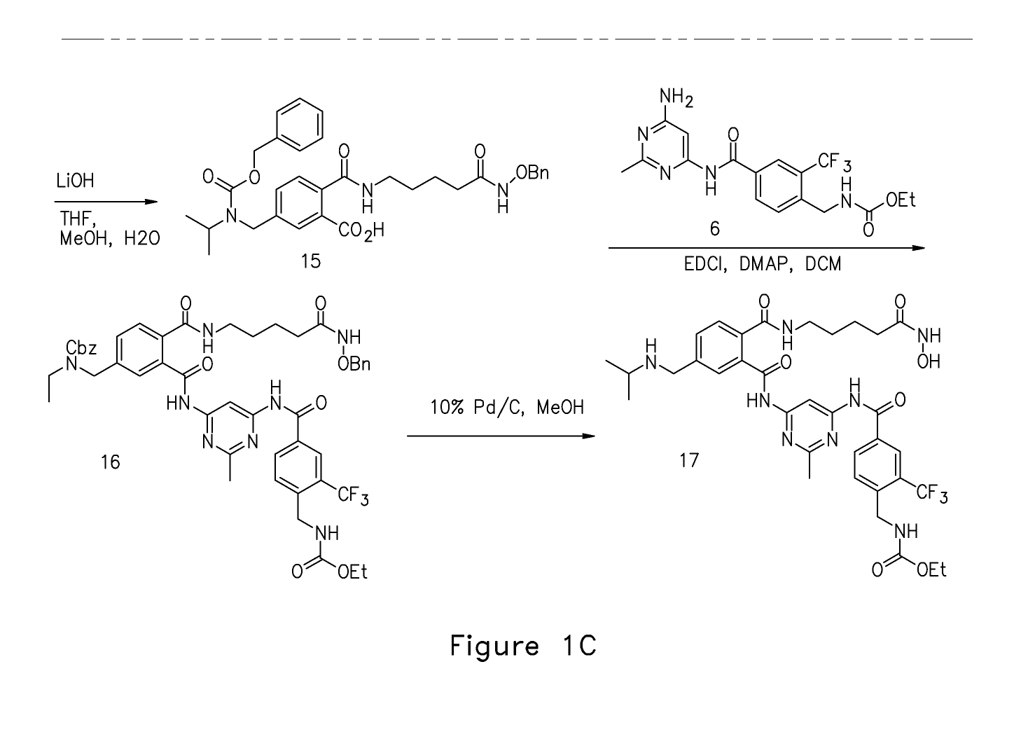

### DETAILED DESCRIPTION

Several compounds, synthesis, composition, methods and uses thereof as pharmaceutically and therapeutically effective compounds are described herein. Although the present embodiments have been described with reference to specific example embodiments, it will be evident that various modifications and changes may be made to these embodiments without departing from the broader spirit and scope of the various embodiments.

### DEFINITIONS

As used herein, the following terms and phrases shall have the meanings set forth below. Unless defined otherwise, all technical and scientific terms used herein have the same meaning as commonly understood to one of ordinary skill in the art.

The term “alkyl” refers to the radical of saturated aliphatic groups, including straight-chain alkyl groups, branched-chain alkyl groups, cycloalkyl (alicyclic) groups, alkyl-substituted cycloalkyl groups, and cycloalkyl-substituted alkyl groups. In preferred embodiments, a straight chain or branched chain alkyl has 30 or fewer carbon atoms in its backbone (e.g., C₁-C₃₀ for straight chains, C₃-C₃₀ for branched chains), and more preferably 20 or fewer. Likewise, preferred cycloalkyls have from 3-10 carbon atoms in their ring structure, and more preferably have 5, 6 or 7 carbons in the ring structure.

The term “alkyl” as used herein refers to a saturated linear or branched-chain monovalent hydrocarbon radical of one to twelve carbon atoms. Examples of alkyl groups include, but are not limited to, methyl (Me, —CH₃), ethyl (Et, —CH₂CH₃), 1-propyl (n-Pr, n-propyl, —CH₂CH₂CH₃), 2-propyl (i-Pr, i-propyl, —CH(CH₃)₂), 1-butyl (n-Bu, n-butyl, —CH₂CH₂CH₂CH₃), 2-methyl-1-propyl (i-Bu, i-butyl, —CH₂CH(CH₃)₂), 2-butyl (s-Bu, s-butyl, —CH(CH₃)CH₂CH₃), 2-methyl-2-propyl (t-Bu, t-butyl, —C(CH₃)₃), 1-pentyl (n-pentyl, —CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), 1-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), 3,3-dimethyl-2-butyl (—CH(CH₃)C(CH₃)₃, 1-heptyl, 1-octyl, and the like.

Moreover, the term “alkyl” (or “lower alkyl”) as used throughout the specification, examples, and claims is intended to include both “unsubstituted alkyls” and “substituted alkyls”, the latter of which refers to alkyl moieties having substituents replacing a hydrogen on one or more carbons of the hydrocarbon backbone. Such substituents, if not otherwise specified, can include, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate. For instance, the substituents of a substituted alkyl may include substituted and unsubstituted forms of amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like. Exemplary substituted alkyls are described below. Cycloalkyls can be further substituted with alkyls, alkenyls, alkoxys, alkylthios, aminoalkyls, carbonyl-substituted alkyls, —CF₃, —CN, and the like.

Moreover, such substituents include all those contemplated for alkyl groups, as discussed below, except where stability is prohibitive. For example, substitution of alkenyl groups by one or more alkyl, carbocyclyl, aryl, heterocyclyl, or heteroaryl groups is contemplated.

The term “alkenyl” refers to linear or branched-chain monovalent hydrocarbon radical of two to twelve carbon atoms with at least one site of unsaturation, i.e., a carbon-carbon, sp double bond, wherein the alkenyl radical includes radicals having “cis” and “trans” orientations, or alternatively, “E” and “Z” orientations. Examples include, but are not limited to, ethylenyl or vinyl (—CH═CH₂), allyl (—CH₂CH═CH₂), and the like. The term “alkynyl” refers to a linear or branched monovalent hydrocarbon radical of two to twelve carbon atoms with at least one site of unsaturation, i.e., a carbon-carbon, sp triple bond. Examples include, but are not limited to, ethynyl (—C≡CH), propynyl (propargyl, —CH₂C≡CH), and the like.

The term “acyl” is art-recognized and refers to a group represented by the general formula hydrocarbylC(O)—, preferably alkylC(O)—.

“Aryl” means a monocyclic or polycyclic ring assembly wherein each ring is aromatic or when fused with one or more rings forms an aromatic ring assembly. If one or more ring atoms is not carbon (e.g., N, S), the aryl is a heteroaryl. Cₓ aryl and C_{x-Y} aryl are typically used where X and Y indicate the number of carbon atoms in the ring.

The term “acylamino” is art-recognized and refers to an amino group substituted with an acyl group and may be represented, for example, by the formula hydrocarbyl C(O)NH—.

The term “acylalkyl” is art-recognized and refers to an alkyl group substituted with an acyl group and may be represented, for example, by the formula hydrocarbyl C(O)alkyl.

The term “acyloxy” is art-recognized and refers to a group represented by the general formula hydrocarbylC(O)O—, preferably alkylC(O)O—.

The term “alkoxy” refers to an alkyl group, preferably a lower alkyl group, having an oxygen attached thereto. Representative alkoxy groups include methoxy, ethoxy, propoxy, tert-butoxy and the like.

The term “alkoxyalkyl” refers to an alkyl group substituted with an alkoxy group and may be represented by the general formula alkyl-O-alkyl.

The term “alkenyl”, as used herein, refers to an aliphatic group containing at least one double bond and is intended to include both “unsubstituted alkenyls” and “substituted alkenyls”, the latter of which refers to alkenyl moieties having substituents replacing a hydrogen on one or more carbons of the alkenyl group. Such substituents may occur on one or more carbons that are included or not included in one or more double bonds.

The term “alkylamino”, as used herein, refers to an amino group substituted with at least one alkyl group.

The term “alkylthio”, as used herein, refers to a thiol group substituted with an alkyl group and may be represented by the general formula alkylS—.

The term “alkynyl”, as used herein, refers to an aliphatic group containing at least one triple bond and is intended to include both “unsubstituted alkynyls” and “substituted alkynyls”, the latter of which refers to alkynyl moieties having substituents replacing a hydrogen on one or more carbons of the alkynyl group. Such substituents may occur on one or more carbons that are included or not included in one or more triple bonds. Moreover, such substituents include all those contemplated for alkyl groups, as discussed above, except where stability is prohibitive. For example, substitution of alkynyl groups by one or more alkyl, carbocyclyl, aryl, heterocyclyl, or heteroaryl groups is contemplated.

The term “ether”, as used herein, refers to a hydrocarbyl group linked through an oxygen to another hydrocarbyl group. Accordingly, an ether substituent of a hydrocarbyl group may be hydrocarbyl-O—. Ethers may be either symmetrical or unsymmetrical. Examples of ethers include, but are not limited to, heterocycle-O-heterocycle and aryl-O-heterocycle. Ethers include “alkoxyalkyl” groups, which may be represented by the general formula alkyl-O-alkyl.

The terms “halo” and “halogen” as used herein means halogen and includes chloro, fluoro, bromo, and iodo.

The terms “hetaralkyl” and “heteroaralkyl”, as used herein, refers to an alkyl group substituted with a hetaryl group.

The term “heteroalkyl”, as used herein, refers to a saturated or unsaturated chain of carbon atoms and at least one heteroatom, wherein no two heteroatoms are adjacent.

The terms “heteroaryl” and “hetaryl” include substituted or unsubstituted aromatic single ring structures, preferably 5- to 7-membered rings, more preferably 5- to 6-membered rings, whose ring structures include at least one heteroatom, preferably one to four heteroatoms, more preferably one or two heteroatoms. The terms “heteroaryl” and “hetaryl” also include polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, and pyrimidine, and the like.

The term “heteroatom” as used herein means an atom of any element other than carbon or hydrogen. Preferred heteroatoms are nitrogen, oxygen, and sulfur.

The terms “heterocyclyl”, “heterocycle”, and “heterocyclic” refer to substituted or unsubstituted non-aromatic ring structures, preferably 3- to 10-membered rings, more preferably 3- to 7-membered rings, whose ring structures include at least one heteroatom, preferably one to four heteroatoms, more preferably one or two heteroatoms. The terms “heterocyclyl” and “heterocyclic” also include polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocyclyl groups include, for example, piperidine, piperazine, pyrrolidine, morpholine, lactones, lactams, and the like.

The term “heterocyclylalkyl”, as used herein, refers to an alkyl group substituted with a heterocycle group.

The term “ketone” is art-recognized and may be represented, for example, by the formula C(O)R₉, wherein R₉ represents a hydrocarbyl group.

The compounds of the present disclosure can be present in the form of pharmaceutically acceptable salts. The compounds of the present disclosure can also be present in the form of pharmaceutically acceptable esters (i.e., the methyl and ethyl esters of the acids of formula I may be used as prodrugs). The compounds of the present disclosure can also be solvated, i.e. hydrated. The solvation can be effected in the course of the manufacturing process or can take place i.e. as a consequence of hygroscopic properties of an initially anhydrous compound of formula I (hydration).

Compounds that have the same molecular formula but differ in the nature or sequence of bonding of their atoms or the arrangement of their atoms in space are termed “isomers.” Isomers that differ in the arrangement of their atoms in space are termed “stereoisomers.” Diastereomers are stereoisomers with opposite configuration at one or more chiral centers which are not enantiomers. Stereoisomers bearing one or more asymmetric centers that are non-superimposable mirror images of each other are termed “enantiomers.” When a compound has an asymmetric center, for example, if a carbon atom is bonded to four different groups, a pair of enantiomers is possible. An enantiomer can be characterized by the absolute configuration of its asymmetric center or centers and is described by the R- and S-sequencing rules of Cahn, lngold and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”. The instant compound of formula I may be used in above mentioned configurations.

The term “sulfate” is art-recognized and refers to the group OSO₃H, or a pharmaceutically acceptable salt thereof. The term “polymorph” as used herein is art-recognized and refers to one crystal structure of a given compound.

“Residue” is an art-recognized term that refers to a portion of a molecule. For instance, a residue of compounds or pharmaceutically acceptable salts of formula I.

The phrases “parenteral administration” and “administered parenterally” as used herein refer to modes of administration along with enteral and topical administration, such as injections, and include without limitation intravenous, intramuscular, intrapleural, intravascular, intrapericardial, intraarterial, intrathecal, intracapsular, intraorbital, intracardiac, intradennal, intraperitoneal, transtracheal, subcutaneous, subcuticular, intra-articular, subcapsular, subarachnoid, intraspinal and intrasternal injection peroral, topical, transdermal, targeted delivery and sustained release and infusion.

A “mammal”, “patient,” “subject,” or “host” to be treated by the subject method may mean either a human or non-human animal, such as primates, mammals, and vertebrates.

The phrase “pharmaceutically acceptable” is art-recognized. In certain embodiments, the term includes compositions, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” is art-recognized, and includes, for example, pharmaceutically acceptable materials, compositions or vehicles, such as a liquid or solid filler, diluent, solvent or encapsulating material involved in carrying or transporting any subject composition, from one organ, or portion of the body, to another organ, or portion of the body. Each carrier must be “acceptable” in the sense of being compatible with the other ingredients of a subject composition and not injurious to the patient. In certain embodiments, a pharmaceutically acceptable carrier is non-pyrogenic. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) cocoa butter and suppository waxes; (9) oils, such as peanut oil, cottonseed oil, sunflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The term “prodrug” is intended to encompass compounds that, under physiological conditions, are converted into the therapeutically active agents of the present disclosure. A common method for making a prodrug is to include selected moieties that are hydrolyzed under physiological conditions to reveal the desired molecule. In other embodiments, the prodrug is converted by an enzymatic activity of the host animal.

The term “prophylactic or therapeutic” treatment is art-recognized and includes administration to the host of one or more of the subject compositions. If it is administered prior to clinical manifestation of the unwanted condition (e.g., disease or other unwanted state of the host animal) then the treatment is prophylactic, i.e., it protects the host against developing the unwanted condition, whereas if it is administered after manifestation of the unwanted condition, the treatment is therapeutic, (i.e., it is intended to diminish, ameliorate, or stabilize the existing unwanted condition or side effects thereof).

The term “treating” is art-recognized and includes preventing a disease, disorder or condition from occurring in an animal which may be predisposed to the disease, disorder and/or condition but has not yet been diagnosed as having it; inhibiting the disease, disorder or condition, e.g., impeding its progress; and relieving the disease, disorder, or condition, e.g., causing regression of the disease, disorder and/or condition. Treating the disease or condition includes ameliorating at least one symptom of the particular disease or condition, even if the underlying pathophysiology is not affected, such as treating the cancer of a subject by administration of an analgesic agent even though such agent does not treat the cause of the cancer. The term “treating”, “treat” or “treatment” as used herein includes curative, preventative (e.g., prophylactic), adjunct and palliative treatment.

Cancer related diseases or disorders includes such as Cancer or malignant neoplasm which include Prostate cancer, Lung cancer, Colon cancer, Breast cancer, Brain cancer, Cervical cancer, Hodgkin's lymphoma, Kidney cancer, Leukemia, Liver cancer, Non-Hodgkin's lymphoma, Ovarian cancer, Skin cancer, Testicular cancer, Thyroid cancer or Uterine cancer or Acute Lymphoblastic Leukemia, Adult Acute Lymphoblastic Leukemia, Childhood Acute Myeloid Leukemia, Adult Acute Myeloid Leukemia, Childhood Adrenocortical Carcinoma, Adrenocortical Carcinoma, Childhood AIDS-Related Cancers, AIDS-Related Lymphoma, Anal Cancer, Appendix Cancer, Astrocytomas, Childhood, Atypical Teratoid/Rhabdoid Tumor, Childhood, Central Nervous System, Basal Cell Carcinoma, see Skin Cancer (Nonmelanoma), Bile Duct Cancer, Extrahepatic Bladder Cancer, Bladder Cancer, Childhood Bone Cancer, Osteosarcoma and Malignant Fibrous Histiocytoma, Brain Stem Glioma, Childhood Brain Tumor, Adult Brain Tumor, Brain Stem Glioma, Childhood Brain Tumor, Central Nervous System Atypical Teratoid/Rhabdoid Tumor, Childhood Brain Tumor, Central Nervous System Embryonal Tumors, Childhood Brain Tumor, Astrocytomas, Childhood Brain Tumor, Craniopharyngioma, Childhood Brain Tumor, Ependymoblastoma, Brain Tumor, Ependymoma, Childhood Brain Tumor, Medulloblastoma, Childhood Brain Tumor, Medulloepithelioma, Brain Tumor, Pineal Parenchymal Tumors of Intermediate Differentiation, Brain Tumor, Supratentorial Primitive Neuroectodermal Tumors and Pineoblastoma, Childhood Brain and Spinal Cord Tumors, Childhood, Breast Cancer, Breast Cancer and Pregnancy, Breast Cancer, Childhood Breast Cancer, Male Bronchial Tumors, Childhood Burkitt Lymphoma. Carcinoid Tumor, Childhood Carcinoid Tumor, Gastrointestinal Carcinoma of Unknown Primary Central Nervous System Atypical Teratoid/Rhabdoid Tumor, Childhood Central Nervous System Embryonal Tumors, Childhood, Central Nervous System Lymphoma, Primary Cervical Cancer Cervical Cancer, Childhood Childhood Cancers Chordoma, Childhood Chronic Lymphocytic Leukemia, Chronic Myelogenous Leukemia, Chronic Myeloproliferative Disorders, Colon Cancer, Colorectal Cancer, Childhood Craniopharyngioma, Childhood Cutaneous T-Cell Lymphoma, Embryonal Tumors, Central Nervous System, Childhood Endometrial Cancer, Ependymoblastoma, Childhood Ependymoma, Childhood Esophageal Cancer, Esophageal Cancer, Childhood Ewing Sarcoma Family of Tumors, Extracranial Germ Cell Tumor, Childhood Extragonadal Germ Cell Tumor, Extrahepatic Bile Duct Cancer, Eye Cancer, Intraocular Melanoma, Eye Cancer, Retinoblastoma, Gallbladder Cancer, Gastric (Stomach) Cancer, Childhood Gastrointestinal Carcinoid Tumor, Gastrointestinal Stromal Tumor (GIST), Gastrointestinal Stromal Cell Tumor, Childhood Germ Cell Tumor, Extracranial, Childhood Germ Cell Tumor, Extragonadal Germ Cell Tumor, Ovarian Gestational Trophoblastic Tumor Glioma, Adult Glioma, Childhood Brain Stem, Hairy Cell Leukemia, Head and Neck Cancer, Hepatocellular (Liver) Cancer, Adult (Primary) Hepatocellular (Liver) Cancer, Childhood (Primary) Histiocytosis, Langerhans Cell Hodgkin Lymphoma, Adult Hodgkin Lymphoma, Childhood Hypopharyngeal Cancer, Intraocular Melanoma, Islet Cell Tumors (Endocrine Pancreas), Kaposi Sarcoma Kidney (Renal Cell) Cancer, Kidney Cancer, Childhood, Langerhans Cell Histiocytosis, Laryngeal Cancer, Childhood Leukemia, Acute Lymphoblastic, Adult Leukemia, Acute Lymphoblastic, Childhood Leukemia, Acute Myeloid, Adult Leukemia, Acute Myeloid, Childhood Leukemia, Chronic Lymphocytic Leukemia, Chronic Myelogenous Leukemia, Hairy Cell Lip and Oral Cavity Cancer Liver Cancer, Adult (Primary) Liver Cancer, Childhood (Primary) Lung Cancer, Non-Small Cell Lung Cancer, Small Cell Lymphoma, AIDS-Related Lymphoma, Burkitt Lymphoma, Cutaneous T-Cell, see Mycosis Fungoides and Sézary Syndrome Lymphoma, Hodgkin, Adult Lymphoma, Hodgkin, Childhood Lymphoma, Non-Hodgkin, Adult Lymphoma, Non-Hodgkin, Childhood Lymphoma, Primary Central Nervous System, Macroglobulinemia, Waldenström Malignant Fibrous Histiocytoma of Bone and Osteosarcoma Medulloblastoma, Childhood Medulloepithelioma, Childhood Melanoma, Melanoma, Intraocular (Eye) Merkel Cell Carcinoma Mesothelioma, Adult Malignant Mesothelioma, Childhood Metastatic Squamous Neck Cancer with Occult Primary Mouth Cancer, Multiple Endocrine Neoplasia Syndrome, Childhood Multiple Myeloma/Plasma Cell Neoplasm, Mycosis Fungoides, Myelodysplastic Syndromes, Myelodysplastic/Myeloproliferative Neoplasms, Myelogenous Leukemia, Chronic Myeloid Leukemia, Adult Acute Myeloid Leukemia, Childhood Acute Myeloma, Multiple Myeloproliferative Disorders, Chronic, Nasal Cavity and Paranasal Sinus Cancer Nasopharyngeal Cancer, Nasopharyngeal Cancer, Childhood Neuroblastoma, Non-Hodgkin Lymphoma, Adult Non-Hodgkin Lymphoma, Childhood Non-Small Cell Lung Cancer, Oral Cancer, Childhood Oral Cavity Cancer, Lip and Oropharyngeal Cancer, Osteosarcoma and Malignant Fibrous Histiocytoma of Bone Ovarian Cancer, Childhood Ovarian Epithelial Cancer, Ovarian Germ Cell Tumor, Ovarian Low Malignant Potential Tumor, Pancreatic Cancer Pancreatic Cancer, Childhood Pancreatic Cancer, Islet Cell Tumors Papillomatosis, Childhood Paranasal Sinus and Nasal Cavity Cancer Parathyroid Cancer Penile Cancer Pharyngeal Cancer Pineal Parenchymal Tumors of Intermediate Differentiation, Childhood Pineoblastoma and Supratentorial Primitive Neuroectodermal Tumors, Childhood Pituitary Tumor Plasma Cell Neoplasm/Multiple Myeloma Pleuropulmonary Blastoma Pregnancy and Breast Cancer Primary Central Nervous System Lymphoma Prostate Cancer, Rectal Cancer Renal Cell (Kidney) Cancer Renal Cell (Kidney) Cancer, Childhood Renal Pelvis and Ureter, Transitional Cell Cancer, Respiratory Tract Carcinoma Involving the NUT Gene on Chromosome 15, Retinoblastoma, Rhabdomyosarcoma, Childhood, Salivary Gland Cancer Salivary Gland Cancer, Childhood Sarcoma, Ewing Sarcoma Family of Tumors Sarcoma, Kaposi Sarcoma, Soft Tissue, Adult Sarcoma, Soft Tissue, Childhood Sarcoma, Uterine Sézary Syndrome, Skin Cancer (Nonmelanoma), Skin Cancer, Childhood Skin Cancer (Melanoma), Skin Carcinoma, Merkel Cell Small Cell Lung Cancer Small Intestine Cancer Soft Tissue Sarcoma, Adult Soft Tissue Sarcoma, Childhood Squamous Cell Carcinoma, see Skin Cancer (Nonmelanoma) Squamous Neck Cancer with Occult Primary, Metastatic Stomach (Gastric) Cancer Stomach (Gastric) Cancer, Childhood Supratentorial Primitive Neuroectodermal Tumors, Childhood. T-Cell Lymphoma, Cutaneous, see Mycosis Fungoides and Sézary Syndrome Testicular Cancer Throat Cancer Thymoma and Thymic Carcinoma Thymoma and Thymic Carcinoma, Childhood Thyroid Cancer Thyroid Cancer, Childhood Transitional Cell Cancer of the Renal Pelvis and Ureter Trophoblastic Tumor, Gestational. Vaginal Cancer, Vaginal Cancer, Childhood, Vulvar Cancer, Waldenström Macroglobulinemia, Wilms Tumor, Women's Cancers or any other medical condition, is well understood in the art, and includes administration of a composition which reduces the frequency of, or delays the onset of, symptoms of a medical condition in a subject relative to a subject which does not receive the composition.

In certain embodiments, the pharmaceutical compositions described herein are formulated in a manner such that said compositions will be delivered to a patient in a therapeutically effective amount, as part of a prophylactic or therapeutic treatment. The desired amount of the composition to be administered to a patient will depend on absorption, inactivation, and excretion rates of the drug as well as the delivery rate of the salts and compositions from the subject compositions. It is to be noted that dosage values may also vary with the severity of the condition to be alleviated. It is to be further understood that for any particular subject, specific dosage regimens should be adjusted over time according to the individual need and the professional judgment of the person administering or supervising the administration of the compositions. Typically, dosing will be determined using techniques known to one skilled in the art.

Additionally, the optimal concentration and/or quantities or amounts of any particular salt or composition may be adjusted to accommodate variations in the treatment parameters. Such treatment parameters include the clinical use to which the preparation is put, e.g., the site treated, the type of patient, e.g., human or non-human, adult or child, and the nature of the disease or condition.

In certain embodiments, the dosage of the subject compositions provided herein may be determined by reference to the plasma concentrations of the therapeutic composition or other encapsulated materials. For example, the maximum plasma concentration (Cₘₐₓ) and the area under the plasma concentration-time curve from time 0 to infinity may be used.

When used with respect to a pharmaceutical composition or other material, the term “sustained release” is art-recognized. For example, a subject composition which releases a substance over time may exhibit sustained release characteristics, in contrast to a bolus type administration in which the entire amount of the substance is made biologically available at one time. For example, in particular embodiments, upon contact with body fluids including blood, spinal fluid, mucus secretions, lymph or the like, one or more of the pharmaceutically acceptable excipients may undergo gradual or delayed degradation (e.g., through hydrolysis) with concomitant release of any material incorporated therein, e.g., an therapeutic and/or biologically active salt and/or composition, for a sustained or extended period (as compared to the release from a bolus). This release may result in prolonged delivery of therapeutically effective amounts of any of the therapeutic agents disclosed herein.

The phrases “systemic administration,” “administered systemically,” “peripheral administration” and “administered peripherally” are art-recognized, and include the administration of a subject composition, therapeutic or other material at a site remote from the disease being treated. Administration of an agent directly into, onto, or in the vicinity of cancer sensation of the disease being treated, even if the agent is subsequently distributed systemically, may be termed “local” or “topical” or “regional” administration, other than directly into the central nervous system, e.g., by subcutaneous administration, such that it enters the patient's system and, thus, is subject to metabolism and other like processes.

The phrase “therapeutically effective amount” is an art-recognized term. In certain embodiments, the term refers to an amount of a salt or composition disclosed herein that produces some desired effect at a reasonable benefit/risk ratio applicable to any medical treatment. In certain embodiments, the term refers to that amount necessary or sufficient to eliminate or reduce medical symptoms for a period of time. The effective amount may vary depending on such factors as the disease or condition being treated, the particular targeted constructs being administered, the size of the subject, or the severity of the disease or condition. One of ordinary skill in the art may empirically determine the effective amount of a particular composition without necessitating undue experimentation.

The present disclosure also contemplates prodrugs of the compositions disclosed herein, as well as pharmaceutically acceptable salts of said prodrugs.

This disclosure also discloses a pharmaceutical composition comprising a pharmaceutically acceptable carrier and the composition of a compound of Formula I may be formulated for systemic or topical or oral administration. The pharmaceutical composition may be also formulated for oral administration, oral solution, injection, subdermal administration, or transdermal administration. The pharmaceutical composition may further comprise at least one of a pharmaceutically acceptable stabilizer, diluent, surfactant, filler, binder, and lubricant. A pharmaceutically acceptable compound of formula I is at least one of a tartrate, esylate, mesylate, sulfate salts, hydrate, hydrochloride salt and solvate.

Additionally, the optimal concentration and/or quantities or amounts of any particular compound of formula I may be adjusted to accommodate variations in the treatment parameters. Such treatment parameters include the clinical use to which the preparation is put, e.g., the site treated, the type of patient, e.g., human or non-human, adult or child, and the nature of the disease or condition.

The concentration and/or amount of any compound of formula I may be readily identified by routine screening in animals, e.g., rats, by screening a range of concentration and/or amounts of the material in question using appropriate assays. Known methods are also available to assay local tissue concentrations, diffusion rates of the salts or compositions, and local blood flow before and after administration of therapeutic formulations disclosed herein. One such method is microdialysis, as reviewed by T. E. Robinson et al., 1991, microdialysis in the neurosciences, Techniques, volume 7, Chapter 1. The methods reviewed by Robinson may be applied, in brief, as follows. A microdialysis loop is placed in situ in a test animal. Dialysis fluid is pumped through the loop. When compounds with formula I such as those disclosed herein are injected adjacent to the loop, released drugs are collected in the dialysate in proportion to their local tissue concentrations. The progress of diffusion of the salts or compositions may be determined thereby with suitable calibration procedures using known concentrations of salts or compositions.

In certain embodiments, the dosage of the subject compounds of formula I provided herein may be determined by reference to the plasma concentrations of the therapeutic composition or other encapsulated materials. For example, the maximum plasma concentration (Cₘₐₓ) and the area under the plasma concentration-time curve from time 0 to infinity may be used.

Generally, in carrying out the methods detailed in this disclosure, an effective dosage for the compounds of Formula I is in the range of about 0.3 mg/kg/day to about 60 mg/kg/day in single or divided doses, for instance 1 mg/kg/day to about 50 mg/kg/day in single or divided doses. The compounds of Formulas I may be administered at a dose of, for example, less than 2 mg/kg/day, 5 mg/kg/day, 10 mg/kg/day, 20 mg/kg/day, 30 mg/kg/day, or 40 mg/kg/day. Compounds of Formula I may also be administered to a human patient at a dose of, for example, between 50 mg and 1000 mg, between 100 mg and 800 mg, or less than 1000, 900, 800, 700, 600, 500, 400, 300, 200, or 100 mg per day. In certain embodiments, the compositions herein are administered at an amount that is less than 95%, 90%, 80%, 70%, 60%, 50%, 40%, 30%, 20%, or 10% of the compound of formula I required for the same therapeutic benefit.

For parenteral administration, solutions of the compositions may be prepared in (for example) sesame or peanut oil, aqueous propylene glycol, or in sterile aqueous solutions may be employed. Such aqueous solutions should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, the sterile aqueous media employed are all readily available by standard techniques known to those skilled in the art.

Generally, a composition as described herein may be administered orally, or parenterally (e.g., intravenous, intramuscular, subcutaneous or intramedullary). Topical administration may also be indicated, for example, where the patient is suffering from gastrointestinal disorder that prevent oral administration, or whenever the medication is best applied to the surface of a tissue or organ as determined by the attending physician. Localized administration may also be indicated, for example, when a high dose is desired at the target tissue or organ. For buccal administration the active composition may take the form of tablets or lozenges formulated in a conventional manner.

For purposes of transdermal (e.g., topical) administration, dilute sterile, aqueous or partially aqueous solutions (usually in about 0.1% to 5% concentration), otherwise similar to the above parenteral solutions, may be prepared.

Ophthalmic formulations, eye ointments, powders, solutions and the like, are also contemplated as being within the scope of the disclosures herein.

In solid dosage forms for oral administration (capsules, tablets, pills, dragees, powders, granules and the like), the subject composition is mixed with one or more pharmaceutically acceptable carriers and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using lactose or milk sugars, as well as high molecular weight polyethylene glycols and the like.

A tablet may be made by compression or molding, optionally with one or more accessory ingredients. Compressed tablets may be prepared using a binder (for example, gelatin or hydroxypropylmethyl cellulose), lubricant, inert diluent, preservative, disintegrant (for example, sodium starch glycolate or cross-linked sodium carboxymethyl cellulose), surface-altering or dispersing agent. Molded tablets may be made by molding in a suitable machine a mixture of the subject composition moistened with an inert liquid diluent. Tablets, and other solid dosage forms, such as dragees, capsules, pills and granules, may optionally be scored or prepared with coatings and shells, such as enteric coatings and other coatings well known in the pharmaceutical-formulating art.

Tablets may be plain, film or sugar coated, bisected, embossed, layered or sustained-release. They can be made in a variety of sizes, shapes and colors. Tablets may be swallowed, chewed or dissolved in the buccal cavity or beneath the tongue. They may be dissolved in water for local or topical disclosure. Sterile tablets are normally used for parenteral solutions and for implantation beneath the skin.

In addition to the active or therapeutic ingredients, tablets may contain a number of inert materials known as excipients. They may be classified according to the role they play in the final tablet. The primary composition may include one or more of a filler, binder, lubricant and glidant. Other excipients which give physical characteristics to the finished tablet are coloring agents, and flavors (especially in the case of chewable tablets). Without excipients most drugs and pharmaceutical ingredients cannot be directly-compressed into tablets. This is primarily due to the poor flow and cohesive properties of most drugs. Typically, excipients are added to a formulation to impart good flow and compression characteristics to the material being compressed. Such properties are imparted through pretreatment steps, such as wet granulation, slugging, spray drying spheronization or crystallization.

Lubricants are typically added to prevent the tableting materials from sticking to punches, minimize friction during tablet compression, and allow for removal of the compressed tablet from the die. Such lubricants are commonly included in the final tablet mix in amounts usually of about 1% by weight.

Other desirable characteristics of excipients include the following: high-compressibility to allow strong tablets to be made at low compression forces; impart cohesive qualities to the powdered material; acceptable rate of disintegration; good flow properties that can improve the flow of other excipients in the formula; and cohesiveness (to prevent tablet from crumbling during processing, shipping and handling).

In some cases, it may be desirable to administer in the form of a kit, it may comprise a container for containing the separate compositions such as a divided bottle or a divided foil packet. Typically the kit comprises directions for the administration of the separate components. The kit form is particularly advantageous when the separate components are preferably administered in different dosage forms (e.g., oral and parenteral), are administered at different dosage intervals, or when titration of the individual components of the combination is desired by the prescribing physician.

An example of such a kit is a so-called blister pack. Blister packs are well known in the packaging industry and are widely used for the packaging of pharmaceutical unit dosage forms (tablets, capsules, and the like). Blister packs generally consist of a sheet of relatively stiff material covered with a foil of a plastic material that may be transparent. During the packaging process recesses are formed in the plastic foil. The recesses have the size and shape of the tablets or capsules to be packed. Next, the tablets or capsules are placed in the recesses and the sheet of relatively stiff material is sealed against the plastic foil at the face of the foil which is opposite from the direction in which the recesses were formed. As a result, the tablets or capsules are sealed in the recesses between the plastic foil and the sheet. In some embodiments the strength of the sheet is such that the tablets or capsules can be removed from the blister pack by manually applying pressure on the recesses whereby an opening is formed in the sheet at the place of the recess. The tablet or capsule can then be removed via said opening.

### Methods and Compositions for the Treatment of Cancer:

In one embodiment, a method of treating cancer or related complications, comprising administering to a patient in need thereof a therapeutically effective amount of compound of Formula I:

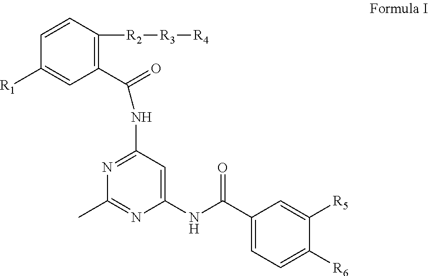

Wherein, R¹, R², R³, R⁴, R⁵, R⁶ each independently represent at least one of hydrogen, methyl, amine, cyclohexyl methyl ether, butoxy, propoxy, halogen (Chlorine or Flourine), thiol, alkyl, alkyl thiol, acetyl thiol, disulfide, acyl, acylalkyl, alkenyl, alkylthioalkyl, alkynyl, alkoxyaryl, alkoxyalkyl, aryl, aralkyl, aryloxyalkyl, arylthioalkyl, cycloalkyl, ether, ester, heteroaryl, heterocyclyl, lower alkyl, sulfone, sulfoxide, hydroxyalkyl, CF₃, CH₂Cl

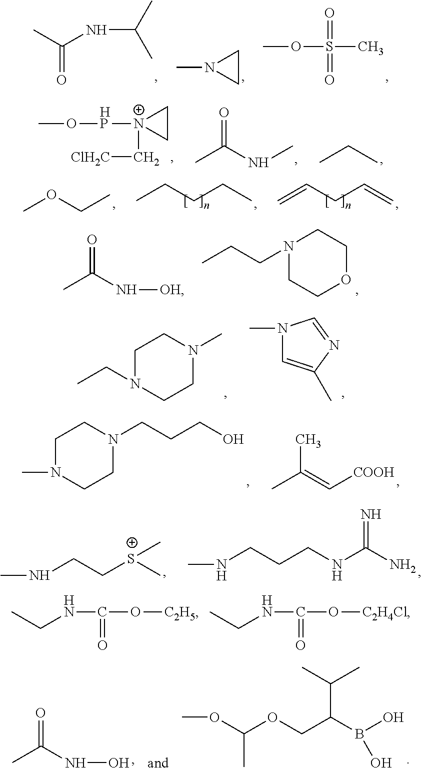

### Method of Synthesis:

STEP-1: To a stirred solution of compound-1 (1.0 eq) in 20 Volumes of methanol and added few drops of sulfuric acid (1.1 eq) was added. Reaction mixture refluxed for 12 h. The reaction mixture quenched with barium carbonate and filtered to remove barium sulfate and filtrate concentrated under reduced pressure. Crude residue 1a was purified by silica gel chromatography.

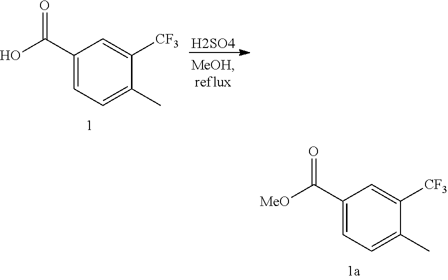

Results:

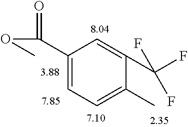

TABLE 1
¹H NMR (CDCl_{3,} 400 MHz)

| | splitting pattern | | |
|---|---|---|---|
| δ | and J value | Protons | Group |
| 8.04 | d | 1H | ArH |
| 7.85 | m | 1H | ArH |
| 7.10 | d | 1H | ArH |
| 2.3 | s | 3H | CH₃ |
| 3.88 | s | 3H | OMe |

STEP-2: To a stirred solution of compound-1a (1.0 eq) in 20 Vol of carbon tetrachloride and added N-bromosuccinimide (1.1 eq) followed by catalytic amount of benzoyl peroxide (0.1 eq). Reaction mixture refluxed for 24 h. The reaction mixture filtered to remove succinamide and filtrate washed with brine, dried over anhydrous Na₂SO₄ and evaporated under reduced pressure. Crude residue purified by silica gel chromatography.

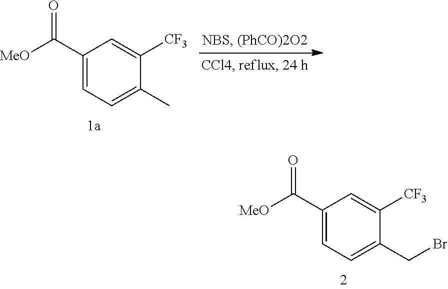

Results:

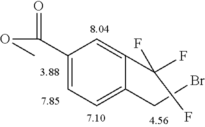

TABLE 2
¹H NMR (CDCl₃, 400 MHz)

| δ | splitting pattern and J value | Protons | Group |
|---|---|---|---|
| 8.04 | d | 1 H | ArH |
| 7.85 | m | 1 H | ArH |
| 7.10 | d | 1 H | ArH |
| 4.56 | s | 2 H | CH₂Br |
| 3.88 | s | 3 H | OMe |

STEP-3: Compound-2 to 3 is a two step procedures, in first step replacement of bromo by amine using methanolic anmonia. In the second step isolated amine was dissolved in DCM and treated with ethyl chloro acetate in presence of triethylamine.

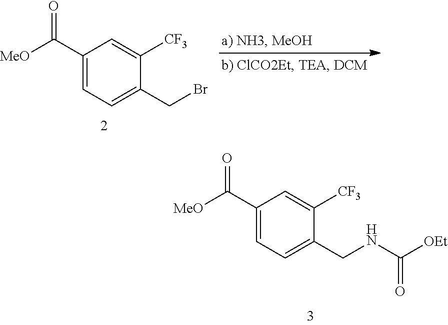

Results:

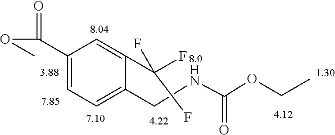

TABLE 3
¹H NMR (CDCl₃, 400 MHz)

| δ | splitting pattern and J value | Protons | Group |
|---|---|---|---|
| 8.04 | d | 1 H | ArH |
| 7.85 | m | 1 H | ArH |
| 7.10 | d | 1 H | ArH |
| 4.22 | s | 2 H | CH₂N |
| 4.12 | q | 2 H | OCH₂CH₃ |
| 3.88 | s | 3 H | OMe |
| 1.30 | t | 3 H | CH₃ |

STEP-4: Compound-3 was dissolved in a mixture of tetrahydrofuran and methanol (1:1), then 1.2 eq of Lithium hydroxide in water added and stirred overnight. Reaction was neutralized with citric acid and the precipitated product was filtered.

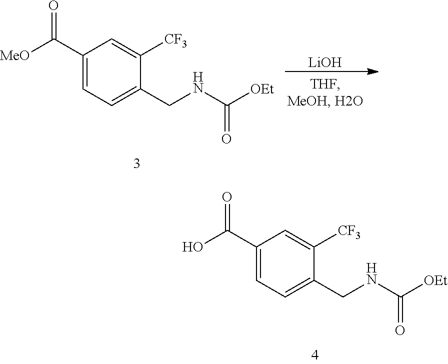

Results:

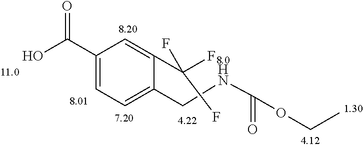

TABLE 4
¹H NMR (CDCl₃, 400 MHz)

| δ | splitting pattern and J value | Protons | Group |
|---|---|---|---|
| 8.20 | d | 1 H | ArH |
| 8.01 | m | 1 H | ArH |
| 7.20 | d | 1 H | ArH |
| 4.22 | s | 2 H | CH₂N |
| 4.12 | q | 2 H | OCH₂CH₃ |
| 1.30 | t | 3 H | CH₃ |

STEP-5: To a stirred solution of compound 4 (1.0 eq) and 6-chloro-2 methyl pyrimidine-4 amine (1.0 eq) in THF (500 mL; LR grade), EDC.HCl (5.2 g, 2.7 mmol; 1.2 eq) and DMAP (3.3 g, 2.7 mmol; 1.2 eq) and followed by 1-hydroxy benzotrizole were added at rt and the reaction mixture was allowed to stir for 16 h at rt. Upon completion of the reaction, distillation with THF under reduced pressure was carried out and the reaction mixture was diluted with DCM (400 mL), washed with water (2×300 mL) followed by brine solution (300 mL) and dried over anhydrous Na₂SO₄ and evaporated under reduced pressure. The crude was purified by column chromatography

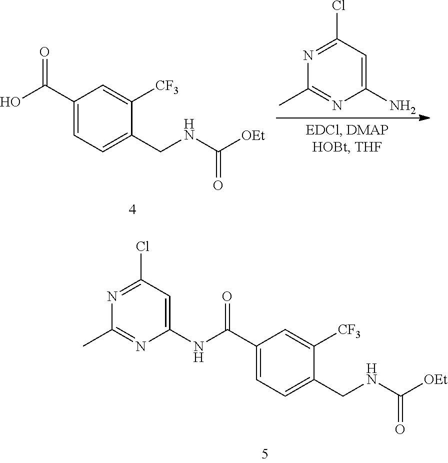

Results:

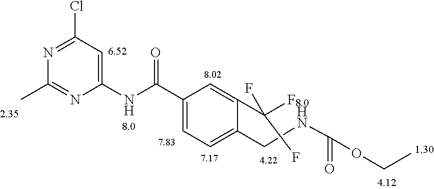

TABLE 5
¹H NMR (CDCl₃, 400 MHz)

| δ | splitting pattern and J value | Protons | Group |
|---|---|---|---|
| 8.02 | d | 1 H | ArH |
| 7.83 | m | 1 H | ArH |
| 7.17 | d | 1 H | ArH |
| 6.52 | s | 1 H | PyrH |
| 4.22 | s | 2 H | CH₂N |
| 4.12 | q | 2 H | OCH₂CH₃ |
| 2.35 | s | 3 H | PyrCH₃ |
| 1.30 | t | 3 H | CH₃ |

STEP-6: To a stirred solution of compound 5 (1.0 eq) in 10 Vol of isopropanol, added saturated ammonia in isopropanol. The reaction mixture was stirred for 12 h and concentrated under reduced pressure. The residue was purified by neutral alumina.

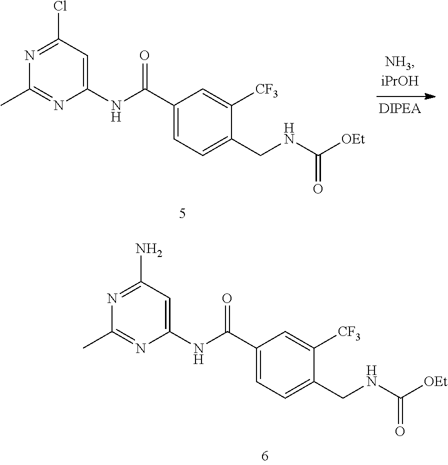

Results:

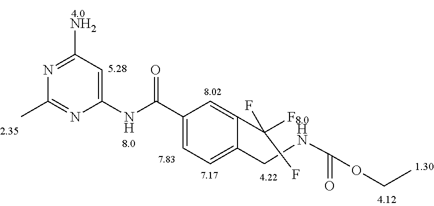

TABLE 6
¹H NMR (CDCl₃, 400 MHz)

| δ | splitting pattern and J value | Protons | Group |
|---|---|---|---|
| 8.02 | d | 1 H | ArH |
| 7.83 | m | 1 H | ArH |
| 7.17 | d | 1 H | ArH |
| 6.52 | s | 1 H | PyrH |
| 4.22 | s | 2 H | CH₂N |
| 4.12 | q | 2 H | OCH₂CH₃ |
| 4.0 | bs | 2 H | NH₂ |
| 2.35 | s | 3 H | PyrCH₃ |
| 1.30 | t | 3 H | CH₃ |

STEP-7: To a stirred solution of compound-7 (1.0 eq) in 20 Vol of carbon tetrachloride and added N-bromosuccinimide (1.1 eq) followed by catalytic amount of benzoyl peroxide (0.1 eq). Reaction mixture was later refluxed for 24 h. The reaction mixture was filtered to remove succinmide and the filtrate was washed with brine, dried over anhydrous Na₂SO₄ and evaporated under reduced pressure. Crude residue was purified by silica gel chromatography

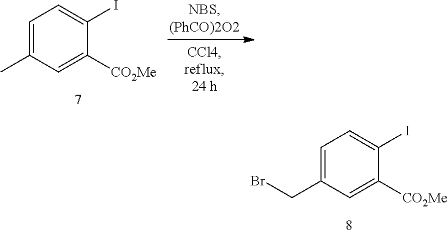

Results:

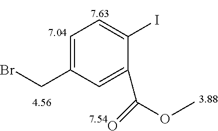

TABLE 7
¹H NMR (CDCl₃, 400 MHz)

| δ | splitting pattern and J value | Protons | Group |
|---|---|---|---|
| 7.63 | d | 1 H | ArH |
| 7.54 | d | 1 H | ArH |
| 7.04 | d | 1 H | ArH |
| 4.56 | s | 2 H | CH₂Br |
| 3.88 | s | 3 H | OCH₃ |

STEP-8: Compound-8 (1.0 eq) was taken in 1.0 eq of isopropyl amine and microwaved for 20 min to provide required compound 9, which was purified by column chromatography.

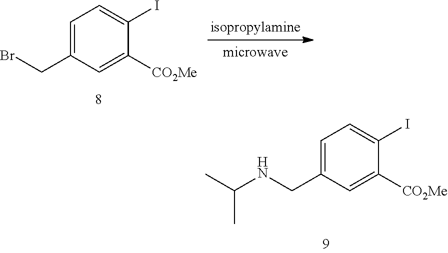

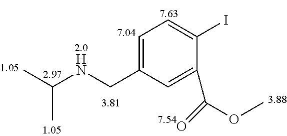

Results:

TABLE 8
¹H NMR (CDCl₃, 400 MHz)

| δ | splitting pattern and J value | Protons | Group |
|---|---|---|---|
| 7.63 | d | 1 H | ArH |
| 7.54 | d | 1 H | ArH |
| 7.04 | d | 1 H | ArH |
| 3.88 | s | 3 H | OCH₃ |
| 3.81 | s | 2 H | CH₂N |
| 2.97 | m | 1 H | CH |
| 1.05 | d | 6 H | CH₃ |

STEP-9: To a stirred solution of compound 9 (1.0 eq) in 10 Vol of dichloromethane, added 2.0 eq of triethylamine and 1.0 eq of benzyloxy chloro formate and reaction mixture stirred overnight. Reaction mixture was quenched with sodium bicarbonate and the compound was extracted with dichloromethane, dried over sodium sulfate, concentrated under reduced pressure and was purified by column chromatography.

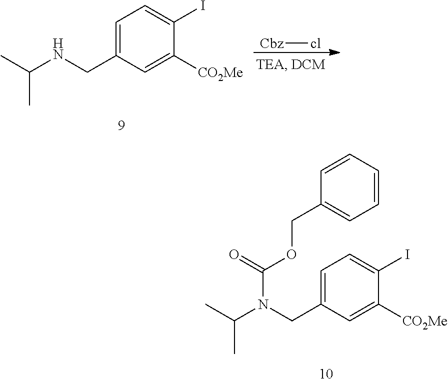

Results:

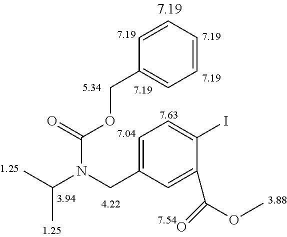

TABLE 9
¹H NMR (CDCl₃, 400 MHz)

| δ | splitting pattern and J value | Protons | Group |
|---|---|---|---|
| 7.63 | d | 1 H | ArH |
| 7.54 | d | 1 H | ArH |
| 7.19 | m | 5 H | OCR₂Ph |
| 7.04 | d | 1 H | ArH |
| 5.34 | s | 2 H | OCH₂ |
| 3.88 | s | 3 H | OCH₃ |
| 4.22 | s | 2 H | CH₂N |
| 3.94 | m | 1 H | CH |
| 1.25 | d | 6 H | CH₃ |

STEP-10: To a stirred solution of compound 10 (1.0 eq) in 10 Vol of dimethylsulfoxide, added 1.0 eq of palladium acetate and 0.1 eq of 1,1′-bis (diphenylphosphanyl) ferrocene (dppf) catalyst and reaction mixture stirred 1 hr, then carbon monoxide passed for 30 min. Reaction mixture filtered and the filtrate was concentrated under reduced pressure and purified by column chromatography

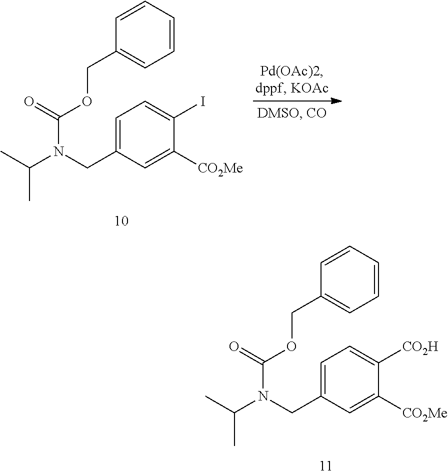

Results:

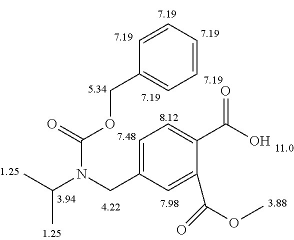

TABLE 10
¹H NMR (CDCl₃, 400 MHz)

| δ | splitting pattern and J value | Protons | Group |
|---|---|---|---|
| 11.0 | bs | 1 H | OH |
| 8.12 | d | 1 H | ArH |
| 7.98 | d | 1 H | ArH |
| 7.48 | d | 1 H | ArH |
| 7.19 | m | 5 H | OCH₂Ph |
| 5.34 | s | 2 H | OCH₂ |
| 4.22 | s | 2 H | CH₂N |
| 3.94 | m | 1 H | CH |
| 3.88 | s | 3 H | OCH₃ |
| 1.25 | d | 6 H | CH₃ |

STEP-11: Compound-12 to 13 conversion is a three step process, in the first step 5-amino pentanoic acid treated with 1.2 eq of boc anhydride in the mixture (1:1, 20 Vol) of THF and water along with 3 eq of sodium carbonate. After 12 h, the reaction mixture was concentrated to remove THF, neutralized with citric acid and extracted with dichloromethane. The obtained Boc derivative of 12 was dissolved in 10 Vol of DCM with 1.0 eq of Triethylamine, 1.0 of benzyloxy amine in presence of coupling agent 1.2 eq of BOP-Cl, coupling reaction completed in 3 h, the isolated product was treated with 1.0 eq of trifluoro acetic acid in 10 Vol of DCM, finally reaction was neutralized with sodium bicarbonate and compound-13 was extracted in dichloromethane.

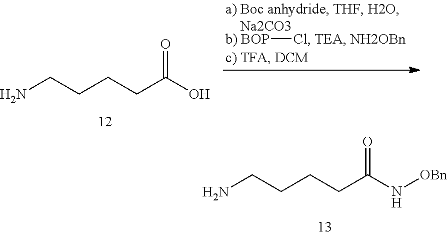

Results:

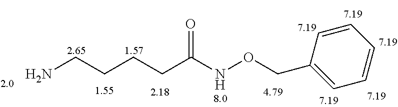

TABLE 11
¹H NMR (CDCl₃, 400 MHz)

| δ | splitting pattern and J value | Protons | Group |
|---|---|---|---|
| 7.19 | m | 5 H | OCH₂Ph |
| 4.79 | s | 2 H | OCH₂ |
| 2.65 | m | 2 H | CH₂NH₂ |
| 2.18 | d | 2 H | CH₂ |
| 1.57 | m | 2 H | CH₂ |
| 1.55 | m | 2 H | CH₂ |

STEP-12: To a stirred solution of compound 11 (1.0 eq) and compound-13 (1.0 eq) in DCM (10 V), EDC.HCl (1.2 eq) and DMAP (1.2 eq) were added at rt and the reaction mixture was allowed to stir for 16 h at rt. On completion of the reaction, the reaction mixture was diluted with DCM (200 mL), washed with water (2×300 mL) followed by brine solution (300 mL) and dried over anhydrous Na₂SO₄ and evaporated under reduced pressure. The crude was purified by column chromatography silica gel to provide compound 14.

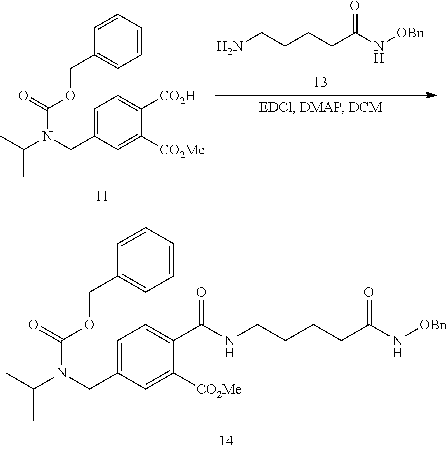

Results:

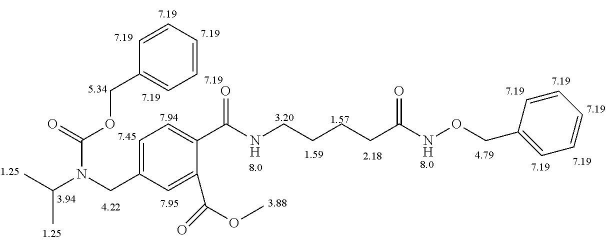

TABLE 12
¹H NMR (CDCl₃, 400 MHz)

| δ | splitting pattern and J value | Protons | Group |
|---|---|---|---|
| 7.95 | s | 1 H | ArH |
| 7.94 | d | 1 H | ArH |
| 7.45 | d | 1 H | ArH |
| 7.19 | m | 10 H | OCH₂Ph |
| 5.34 | s | 2 H | OCH₂ |
| 4.79 | s | 2 H | OCH₂ |
| 4.22 | s | 2 H | NCH₂ |
| 3.94 | m | 1 H | CH |
| 3.88 | s | 3 H | OCH₃ |
| 3.20 | m | 2 H | N CH₂ |
| 2.18 | t | 2 H | COCH₂ |
| 1.59 | m | 2 H | CH₂ |
| 1.57 | m | 2 H | CH₂ |
| 1.25 | d | 6 H | CH₃ |

STEP-13: Compound-14 dissolved in mixture of tetrahydrofuran and methanol (1:1, 10 Vol), then 1.2 eq of Lithium hydroxide in water added and stirred overnight. Reaction neutralized with citric acid and the precipitated product filtered.

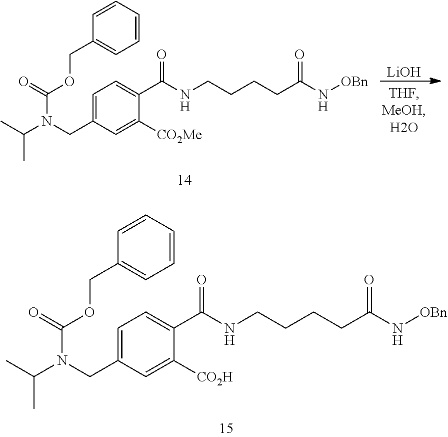

Results:

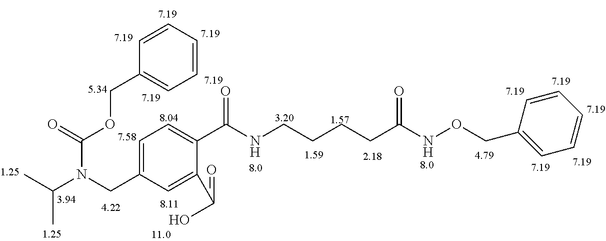

TABLE 13
¹H NMR (CDCl₃, 400 MHz)

| δ | splitting pattern and J value | Protons | Group |
|---|---|---|---|
| 11.0 | s | 1 H | OH |
| 8.11 | s | 1 H | ArH |
| 8.04 | d | 1 H | ArH |
| 7.58 | d | 1 H | ArH |
| 7.19 | m | 10 H | OCH₂Ph |
| 5.34 | s | 2 H | OCH₂ |
| 4.79 | s | 2 H | OCH₂ |
| 4.22 | s | 2 H | NCH₂ |
| 3.94 | m | 1 H | CH |
| 3.20 | m | 2 H | N CH₂ |
| 2.18 | t | 2 H | COCH₂ |
| 1.59 | m | 2 H | CH₂ |
| 1.57 | m | 2 H | CH₂ |
| 1.25 | d | 6 H | CH₃ |

STEP-14: To a stirred solution of compound 15 (1.0 eq) and compound-6 (1.0 eq) in DCM (10 V), EDC.HCl (1.2 eq) and DMAP (1.2 eq) were added at rt and the reaction mixture was allowed to stir for 16 h at rt. On completion of the reaction, the reaction mixture was diluted with DCM (200 mL), washed with water (2×300 mL) followed by brine solution (300 mL) and dried over anhydrous Na₂SO₄ and evaporated under reduced pressure. The crude was purified by column chromatography silica gel to provide compound 16.

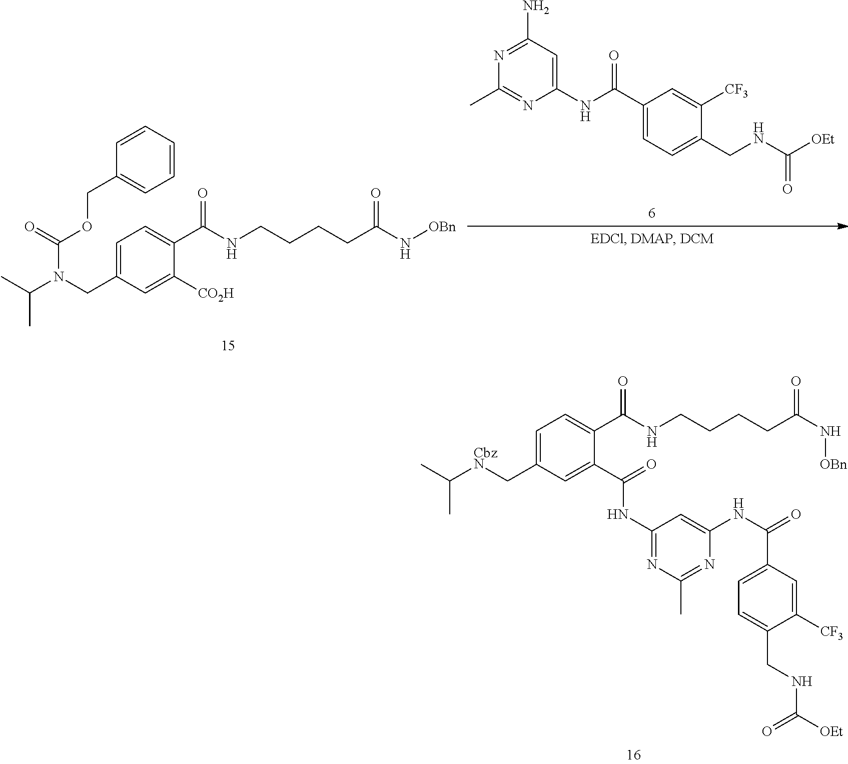

Results:

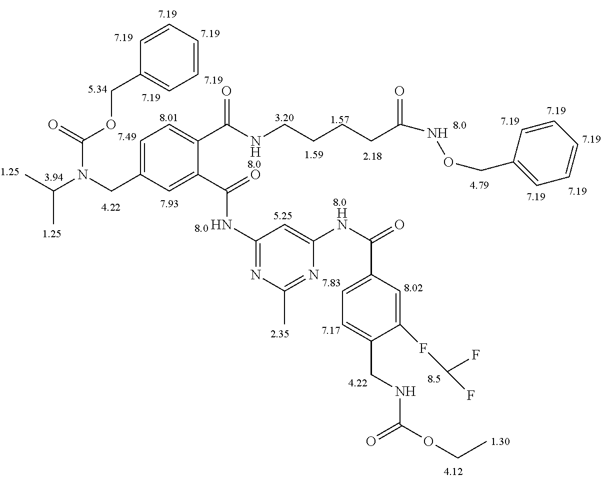

TABLE 14
¹H NMR (CDCl₃, 400 MHz)

| δ | splitting pattern and J value | Protons | Group |
|---|---|---|---|
| 8.02 | s | 1 H | ArH |
| 8.01 | s | 1 H | ArH |
| 7.93 | d | 1 H | ArH |
| 7.83 | d | 1 H | ArH |
| 7.49 | d | 1 H | ArH |
| 7.19 | m | 10 H | OCH₂Ph |
| 7.17 | d | 1 H | ArH |
| 5.34 | s | 2 H | OCH₂ |
| 5.28 | s | 1 H | PyrH |
| 4.79 | s | 2 H | OCH₂ |
| 4.22 | s, s | 4 H | NCH₂ |
| 4.12 | q | 2 H | OCH₂CH₃ |
| 3.94 | m | 1 H | CH |
| 3.20 | m | 2 H | N CH₂ |
| 2.35 | s | 3 H | PyrCH₃ |
| 2.18 | t | 2 H | COCH₂ |
| 1.59 | m | 2 H | CH₂ |
| 1.57 | m | 2 H | CH₂ |
| 1.3 | t | 3 H | OCH₂CH₃ |
| 1.25 | d | 6 H | CH₃ |

STEP-15: To a solution of compound 16 (1.0 eq) in MeOH (400 mL) was added (10%) palladium on carbon (0.2 eq) and stirred under (H₂) balloon pressure for 4 hrs at rt. The reaction mixture was filtered over a celite pad, washed the bed with methanol (300 mL); filtrate was evaporated under the reduced pressure to yield compound 17.

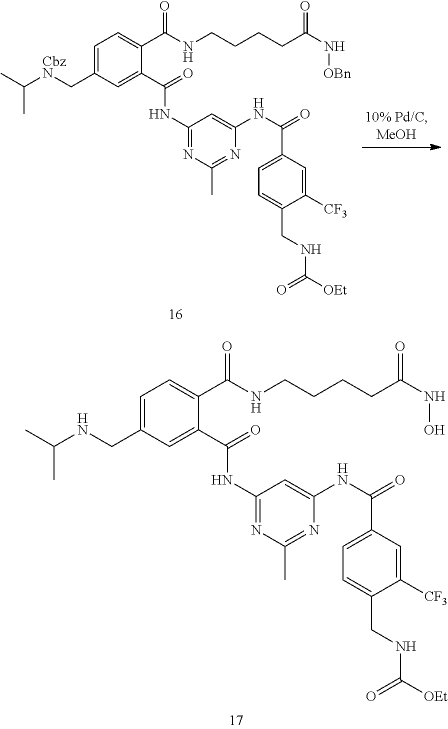

Results:

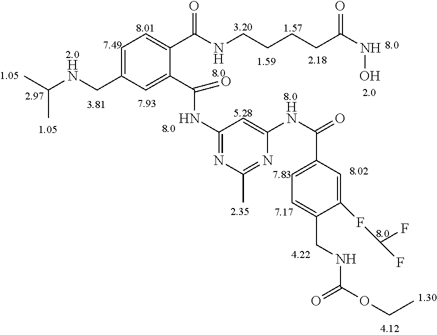

TABLE 15
¹H NMR (CDCl₃, 400 MHz)

| δ | splitting pattern and J value | Protons | Group |
|---|---|---|---|
| 8.02 | s | 1 H | ArH |
| 8.01 | s | 1 H | ArH |
| 7.93 | d | 1 H | ArH |
| 7.83 | d | 1 H | ArH |
| 7.49 | d | 1 H | ArH |
| 7.17 | d | 1 H | ArH |
| 5.28 | s | 1 H | PyrH |
| 4.22 | s, s | 4 H | NCH₂ |
| 4.12 | q | 2 H | OCH₂CH₃ |
| 3.20 | m | 2 H | N CH₂ |
| 2.97 | m | 1 H | CH |
| 2.35 | s | 3 H | PyrCH₃ |
| 2.18 | t | 2 H | COCH₂ |
| 1.59 | m | 2 H | CH₂ |
| 1.57 | m | 2 H | CH₂ |
| 1.3 | t | 3 H | OCH₂CH₃ |
| 1.25 | d | 6 H | CH₃ |

Results:

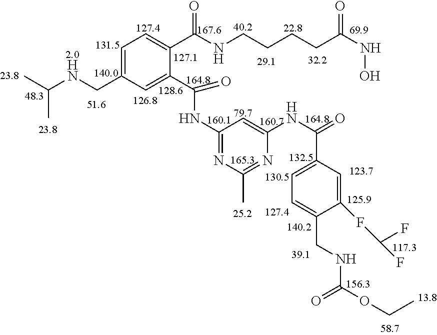

TABLE 16
C13 NMR value:

| δ | |
|---|---|
| 169.9 | 126.8 |
| 167.6 | 125.9 |
| 165.3 | 123.7 |
| 164.8 | 117.3 |
| 164.8 | 79.7 |
| 160.7 | 58.7 |
| 160.1 | 51.6 |
| 156.3 | 48.3 |
| 140.2 | 40.2 |
| 140 | 39.1 |
| 132.5 | 32.2 |
| 131.5 | 29.1 |
| 130.6 | 23.8 |
| 128.6 | 23.8 |
| 127.4 | 22.8 |
| 127.4 | 13.8 |
| 127.1 | |

The present disclosure provides among other things compositions and methods for treating cancer. While specific embodiments of the subject disclosure have been discussed, the above specification is illustrative and not restrictive. Many variations of the systems and methods herein will become apparent to those skilled in the art upon review of this specification. The full scope of the claimed systems and methods should be determined by reference to the claims, along with their full scope of equivalents, and the specification, along with such variations.

We claim:

1. A compound, comprising:
a formula I; 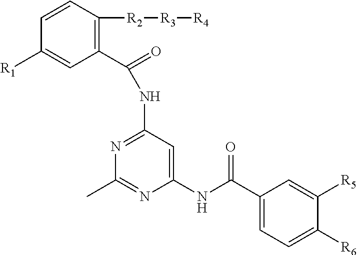
Wherein,
R¹, R², R³, R⁴, R⁵, R⁶ is at least one of hydrogen, methyl, amine, cyclohexyl methyl ether, butoxy, propoxy, halogen (Chlorine or Flourine), thiol, alkyl, alkyl thiol, acetyl thiol, disulfide, acyl, acylalkyl, alkenyl, alkylthioalkyl, alkynyl, alkoxyaryl, alkoxyalkyl, aryl, aralkyl, aryloxyalkyl, arylthioalkyl, cycloalkyl, ether, ester, heteroaryl, heterocyclyl, lower alkyl, sulfone, sulfoxide, hydroxyalkyl, CF₃, CH₂Cl,

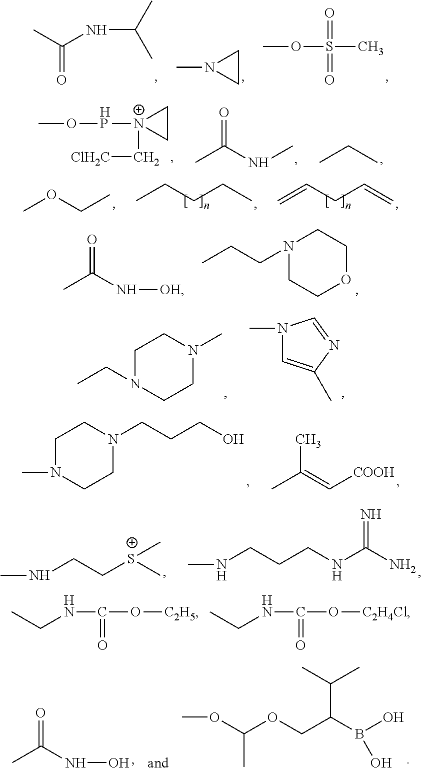

2. The compound of claim 1, further comprising: 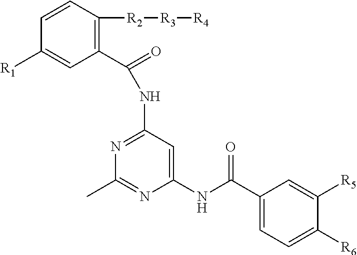
Wherein,
R¹ independently represents at least one of 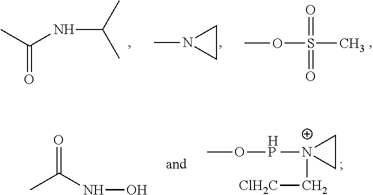
R² independently represents at least one of hydrogen, carboxyl, amine, —NH—CO—NH—, —NH—CO—CH₂—NH—, 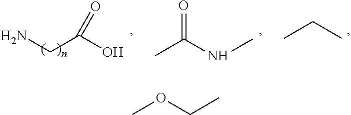
and an analog of any of the foregoing;
wherein, n represents an integer from 0 to 11.
R³ independently represents at least one of hydrogen, methyl, ethyl, butyl, amine, —NH—CO—, acrolein, 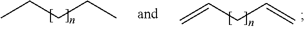
R⁴ independently represents at least one of 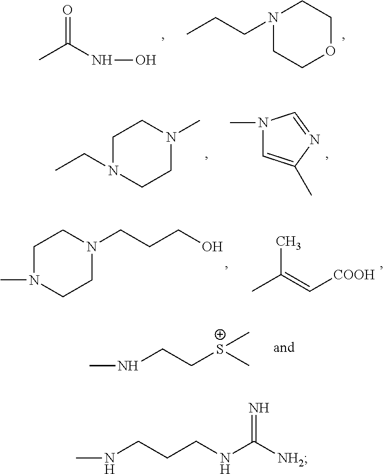
R⁵ independently represents at least one of CF₃, and (CH)ₙCl;
wherein n is an integer ranges from 0 to 11;
R⁶ independently represents at least one of

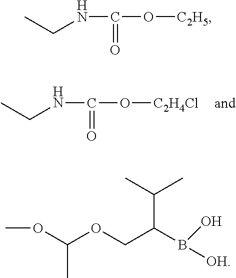

3. The compound of claim 2, further comprising;

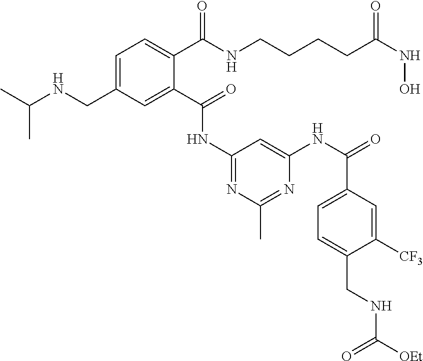

4. The compound of claim 1, further comprising;
a pharmaceutically acceptable compound of formula I is at least one of a tartrate, esylate, mesylate, sulfate salts, hydrate, hydrochloride salt and solvate.

5. The compound of claim 4, wherein administration is at least one of a peroral, topical, transdermal, targeted delivery and sustained release.

6. The compound of claim 4, further comprising:
formula I is formulated for the treatment of cancer.

7. A composition, comprising;
a compound of formula I, a pharmaceutically acceptable stabilizer, a diluent, a surfactant, a filler, a binder, and a lubricant to treat cancer.

8. The composition of claim 7; further comprising:
a compound of formula I, a prodrug of formula I, an isomer, chiral compound, enantinomer and a racemic mixture of formula I.

9. A kit for a compound, comprising:
a compound of formula I in the form of at least one of a tablet, ophthalmic solution, injection and transdermal preparation.

10. The kit of claim 9, further comprising instructions for use in the treatment of cancer and other complications.
